(19) United States
(12) Patent Application Publication
(10) Pub. No.: US 2025/0201423 A1
(43) Pub. Date: Jun. 19, 2025

(54) GENETICS DRIVEN PERSONALIZED DISEASE PROGRESSION MODEL

(71) Applicant: International Business Machines Corporation, Armonk, NY (US)

(72) Inventors: Sanjoy Dey, White Plains, NY (US); Haoyu Yang, Minneapolis, MN (US); Pablo Meyer Rojas, Brooklyn, NY (US); Hongyang Li, Livingston, NJ (US)

(21) Appl. No.: 18/544,872

(22) Filed: Dec. 19, 2023

(51) Int. Cl. G16H 50/50

(52) U.S. Cl. CPC G16H 50/50

(57) ABSTRACT

A method, computer system, and a computer program product for personalized disease progression modeling is provided. The present invention may include receiving patient data, wherein the patient data is comprised of at least genetic data and clinical observations. The present invention may include building a personalized disease progression framework using one or more patient groupings and one or more disease progression pathways. The present invention may include determining a disease stage and progression pattern for each of the one or more patient groupings using a personalizes disease progression model.

### BACKGROUND

The present invention relates generally to the field of computing, and more particularly to personalized disease progression modeling.

Chronic diseases may often progress through multiple stages, often times from mild, to moderate, to severe stages. However, the disease trajectory patterns through these multiple stages are usually not uniform across all patients. The progression patterns of these diseases may often be different from patient to patient due to at least genetics, environmental effects, amongst other factors. Current approaches to disease progression pathways are often not personalized, rather these current approaches often model disease progression as a uniform trajectory pattern on the population level. Modeling disease progression through multiple stages may be critical for clinical decision-making with respect to chronic diseases, the current and/or existing approaches, may often model disease progression as a uniform trajectory pattern at a population level, however, chronic diseases may be highly heterogeneous and may often have multiple progression patterns depending on a patient's individual genetics and environmental effects.

Accordingly, the present invention aims to model the disease progression for unique patient groups separately according to different inherent characteristics defined by genetic compositions which may require a personalized framework for both supervised and unsupervised disease progression models.

### SUMMARY

Embodiments of the present invention disclose a method, computer system, and a computer program product for personalized disease progression modeling. The present invention may include receiving patient data, wherein the patient data is comprised of at least genetic data and clinical observations. The present invention may include building a personalized disease progression framework using one or more patient groupings and one or more disease progression pathways. The present invention may include determining a disease stage and progression pattern for each of the one or more patient groupings using a personalized disease progression model. Accordingly, the present invention improved over the prior art by utilizing a generic framework for modeling diverse chronic diseases irrespective of defined disease states. The personalized disease progression model may utilize unsupervised computational approaches for chronic diseases in which the disease onset may not be well measured and/or staging may be disease onset agnostic. The personalized disease progression model may utilize supervised computational approaches for chronic diseases in which the disease onset may be measured, and the disease may progress through multiple stages.

In another embodiment, the method may include displaying, in a user interface, the disease progression pattern for each of the one or more patient groupings using a personalized disease progression trajectory for each of the one or more patient groupings. Accordingly, the present invention improves over the prior art where progression pathways are often not personalized, but rather they model disease progression as a uniform trajectory pattern on the population model, in contrast, the current invention utilizes a personalized disease progression model to model the progression for each of the one or more patient groupings separately within different inherent characteristics.

In a further embodiment, the method may include monitoring an actual disease progression pattern based on additional patient data received; and comparing the actual disease progression pattern to the personalized disease progression trajectory for a corresponding patient grouping. Accordingly, the present invention improves over the prior art by modeling both static and continuous data together, rather than assuming all data is continuous, while considering the relationships between the static and continuous data. Furthermore, the invention continuously improves upon the personalized disease progression model using additional patient data received.

In yet another embodiment, the method may include generating one or more recommendations for each of the one or more patient groupings based on the disease progression pattern corresponding to each patient grouping, wherein the one or more recommendations includes at least a set of early intervention protocols. Accordingly, the present invention improves is distinguishable from the prior art by designing early intervention protocols based on patient genetics and their disease progression pathways. These early intervention protocols are unique because the personalized progression model considers both static data, such as genome-wide association data, and continuous data, such as clinical observations, such that the early intervention protocols may be specific to each of the one or more patient groupings.

In yet another embodiment, the method may include the personalized disease progression model utilizing a supervised computational approach in leveraging the personalized disease progression framework. In this embodiment the personalized disease progression trajectory includes identifying a current stage from a plurality of disease stages and progression pattern for each of the one or more patient groups. This embodiment illustrates the inventions improvement to progression modeling by building a generic framework for modeling diverse chronic disease irrespective of whether disease states are routinely measured in clinical settings or not.

In addition to a method, additional embodiments are directed to a computer system and a computer program product for building a personalized disease progression model which is flexible enough to be deployed in both unsupervised and supervised disease progression models while modeling both genetic data and clinical observations.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 depicts a block diagram of an exemplary computing environment according to at least one embodiment;

FIG. 2 is an operational flowchart illustrating a process for personalized disease progression modeling according to at least one embodiment;

FIG. 3 depicts a graphical representation of a single disease progression model according to at least one embodiment;

FIG. 4 depicts an architectural representation of the personalized disease progression model according to at least one embodiment;

FIG. 5 depicts a deep learning model for genome-wide association data according to at least one embodiment;

FIG. 6 depicts a personalized disease progression model which may be utilized in at least one embodiment for which a disease onset is unknown; and

FIG. 7 depicts a personalized disease progression model which may be utilized in at least one embodiment for which the disease onset is known.

### DETAILED DESCRIPTION

The following described exemplary embodiments provide a system, method and program product for personalized disease progression modeling. As such, the present embodiment has the capacity to improve the technical field of disease progression modeling by building a personalized disease progression model which is flexible enough to be deployed in both unsupervised and supervised disease progression models while modeling both genetic data and clinical observations. More specifically, the present invention may include receiving patient data, wherein the patient data is comprised of at least genetic data and clinical observations. The present invention may include building a personalized disease progression framework using one or more patient groupings and one or more disease progression pathways. The present invention may include determining a disease stage and progression pattern for each of the one or more patient groupings using a personalizes disease progression model.

As described previously, chronic diseases may often progress through multiple stages, often times from mild, to moderate, to severe stages. However, the disease trajectory patterns through these multiple stages are usually not uniform across all patients. The progression patterns of these diseases may often be different from patient to patient due to at least genetics, environmental effects, amongst other factors. Current approaches to disease progression pathways are often not personalized, rather these current approaches often model disease progression as a uniform trajectory pattern on the population level. Modeling disease progression through multiple stages may be critical for clinical decision-making with respect to chronic diseases, the current and/or existing approaches, may often model disease progression as a uniform trajectory pattern at a population level, however, chronic diseases may be highly heterogeneous and may often have multiple progression patterns depending on a patient's individual genetics and environmental effects.

Accordingly, the present invention aims to model the disease progression for unique patient groups separately according to different inherent characteristics defined by genetic compositions which may require a personalized framework for both supervised and unsupervised disease progression models.

Therefore, it may be advantageous to, among other things, receive patient data, wherein the patient data is comprised of at least genetic data and clinical observations, build a personalized disease progression framework using one or more patient groupings and one or more disease progression pathways, and determine a disease stage and a progression pattern for each of the one or more patient groupings using a personalized disease progression model.

According to at least one embodiment, the present invention may improve disease progression modeling by utilizing a personalized disease progression model to jointly learn the heterogeneous progression patterns and groups of genetic profiles. In particular, an end-to-end pipeline is designed to simultaneously infer the characteristics of patents from genetic markers using a variational autoencoder and how it drives the disease progressions using a Recurrent Neural Network (RNN) based state-space model based on clinical observations.

According to at least one embodiment, the present invention may improve upon current approaches to disease progression pathways are often not personalized, rather these current approaches often model disease progression as a uniform trajectory pattern on the population level. Modeling disease progression through multiple stages may be critical for clinical decision-making with respect to chronic diseases, the current and/or existing approaches, may often model disease progression as a uniform trajectory pattern at a population level, however, chronic diseases may be highly heterogeneous and may often have multiple progression patterns depending on a patient's individual genetics and environmental effects. Accordingly, the present invention aims to model the disease progression for unique patient groups separately according to different inherent characteristics defined by genetic compositions which may require a personalized framework for both supervised and unsupervised disease progression models.

According to at least one embodiment, the present invention may improve disease progression modeling by modeling disease heterogeneity using personalized models which may be defined by genetic markups of patients.

According to at least one embodiment, the present invention may improve disease progression modeling by building a generic framework for modeling diverse chronic diseases irrespective of whether disease states are routinely measured in clinical settings or not.

According to at least one embodiment, the present invention may improve disease progression modeling by developing a method for modeling both static data, such as, but not limited to, genetics data retrieved from the Genome-wide Association (GWA) and continuous data, such as, but not limited to, clinical observations.

According to at least one embodiment, the present invention may improve disease progression modeling by utilizing the relationships between both static and continuous data for jointly learning different patient clusters and personalizing disease progression models defined for each of those clusters.

According to at least one embodiment, the present invention may improve disease progression modeling by discovering different disease progression pathways with potentially elucidating diverse biological mechanisms of a same disease.

According to at least one embodiment, the present invention may improve disease progression modeling by designing early intervention protocols based on patient's genetics and their disease progression pathways.

According to at least one embodiment, the present invention may improve disease progression modeling by building a genetics-driven personalized disease progression model (GWA-PerDPM) (e.g., personalized disease progression model) which may discover diverse genetics groupings based on large-scale genome-wide association (GWA) data which may impact the disease progression automatically using a variational auto-encoder.

According to at least one embodiment, the present invention may improve disease progression modeling by building a GWA-PerDPM (e.g., personalized disease progression model) in which the proposed technique may model disease progression across multiple stages using a state-space based generative model in which the transition between states are dependent on the genetic markup of patients.

According to at least one embodiment, the present invention may improve disease progression modeling by building a GWA-PerDPM (e.g., personalized disease progression model) which may utilize a joint learning optimization framework for inference of multi-dimensional time-varying representation where the clustering of genetic data and the corresponding disease progression model may be performed simultaneously.

According to at least one embodiment, the present invention may improve disease progression modeling by building a GWA-PerDPM (e.g., personalized disease progression model) which may be flexible enough to be deployed in both unsupervised and supervised disease progression models. In particular, the invention may utilize the true state of disease for supervised learning as an additional loss function with the regular (ELBO) based loss.

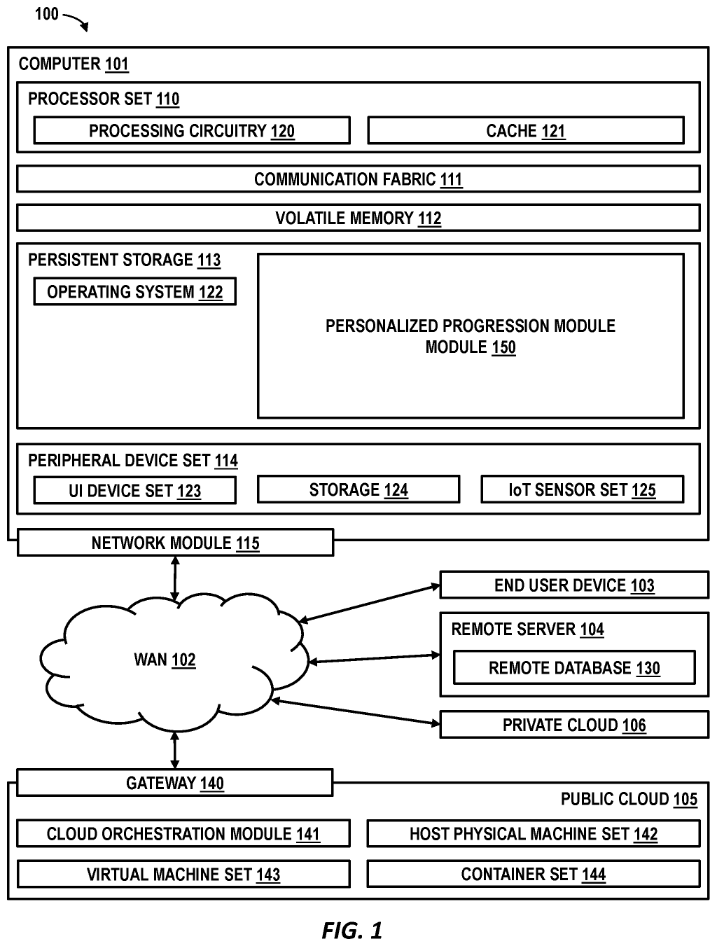

Referring to FIG. 1, Computing environment 100 contains an example of an environment for the execution of at least some of the computer code involved in performing the inventive methods, such as building a personalized disease progression model which is flexible enough to be deployed in both unsupervised and supervised disease progression models while modeling both genetic data and clinical observations using the personalized progression module 150. In addition to module 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and module 150, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor Set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in module 150 in persistent storage 113.

Communication fabric 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent Storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in module 150 typically includes at least some of the computer code involved in performing the inventive methods.

Peripheral device set 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End User Device (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

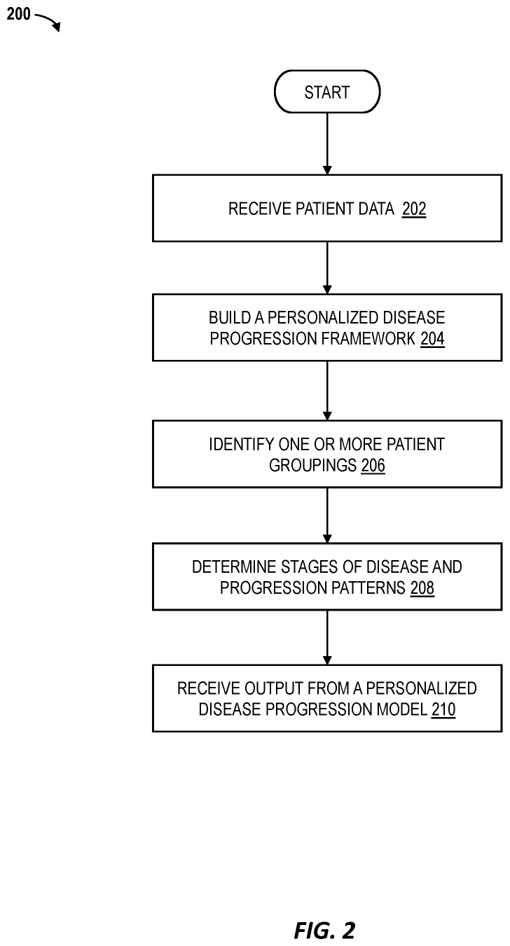

According to the present embodiment, the computer environment 100 may use the personalized progression module 150 to building a personalized disease progression model which is flexible enough to be deployed in both unsupervised and supervised disease progression models while modeling both genetic data and clinical observations. The personalized disease progression modeling method is explained in more detail below with respect to FIGS. 2-7.

Referring now to FIG. 2, an operational flowchart illustrating the exemplary personalized disease progression modeling process 200 used by the personalized progression module 150 according to at least one embodiment is depicted.

At 202, the personalized progression module 150 receives patient data. The patient data received by the personalized progression module 150 may include both clinical observations and genetic data. All patient data received by the personalized progression module 150 shall not be construed as to violate or encourage the violation of any local, state, federal, or international law with respect to privacy protection.

The patient data may be received by the personalized progression module 150 for a plurality of patients selected by a user within a user interface. The user may select the plurality of patients within the user interface based on chronic disease, patient characteristics, and/or other criteria. The user may be an authorized user and/or multiple users associated with an entity, such as, but not limited to, researchers, practitioners, administrators, and/or other medical/health care professionals, the entity may be, for example, a medical institution, academic institution, a pharmaceutical company, healthcare provider or payer, and/or other entity with permittable access to patient data. The personalized progression module 150 may require consent from each of the plurality of patients for which the data may be received prior to receiving the patient data and/or consent from the entity on behalf of the plurality of patients. The patient data may be anonymized prior to the patient data being received by the disease progression module 150 such that re-identification risk may be reduced. Additionally, the personalized progression module 150 may also anonymize the patient data, for example, the personalized progression module 150 may apply perturbations, adversarial patches, and/or other noise to visual patient data, such as, but not limited to, include medical images, medical videos, amongst other visual content, which may include, but is not limited to including Magnetic Resonance Imaging (MRI) scans, Computed Tomography (CT) scans, X-ray images, ultrasounds, Positron Emission Tomography (PET) scans, Arthrograms, Myelograms, amongst other visual patient data. The personalized progression module 150 may only receive data relevant to the specific disease being modeled.

The patient data received by the personalized progression module 150 may be stored in a database 130 which may utilize a private cloud and/or a protected database of the personalized progression module 150 and/or entity in preserving the privacy of the patient data. All of the patient data received may be secured and/or maintained by a cloud based service, such as, but not limited to, IBM Cloud® (IBM Cloud® and all IBM-based trademarks are trademarks or registered trademarks of International Business Machines Corporation in the United States, and/or other countries), amongst other cloud based services. The cloud based service may utilize, public cloud, private cloud, and/or hybrid cloud in securing the patient data.

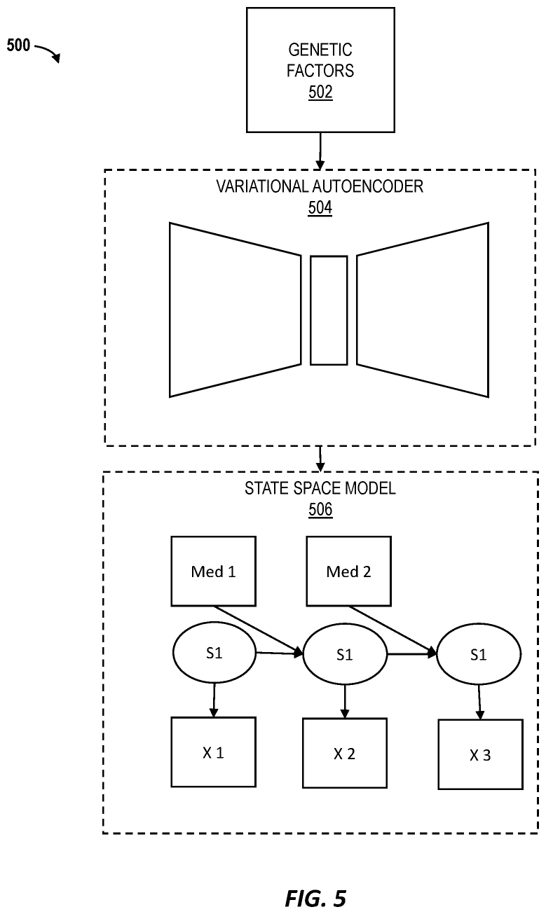

The patient data received by the personalized progression module 150 may include both clinical data (e.g., clinical observations) and genetic data. As will be explained in more detail below, the personalized progression module 150 may utilize both static data (e.g., genetic data) and continuous data (e.g., clinical data) as input for the personalized disease progression model such that the output of the personalized disease progression model accounts for the relationships between the clinical and genetic data. Clinical observations may include, but are not limited to including, electronic health records (EHR), medications, laboratory results and/or data, comorbidities, treatments, amongst other clinical observations or data which may be recorded by the entity, users associated with the entity, and/or data maintained by the entity with respect to the plurality of patients. Genetic data may include data retrieved from large-scale genome-wide association studies (GWAS) data (e.g., GWA data), amongst other genetic data which may be utilized to identify genomic variants that are statistically associated with a risk for a disease or a particular trait. The genetic data which may be collected in GWA studies may measure the genetic variation of an individual (e.g., patient) at a single base position of the individual's Deoxyribonucleic acid (DNA). These genetic variations, which may be referred to as Single Nucleotide Polymorphisms (SNPs), may impact how and/or to what degree a particular trait or disease phenotype may be manifested for an individual (e.g., patient) and to what degree. Accordingly, due to the high-dimensionality and noise which may be associated with GWA, the invention may assume that there may be a few distinct genomic markups for a particular disease which may manifest at different degrees for each individual sample, which will be explained in greater detail below with respect to at least step 204 and FIG. 5. The personalized progression module 150 may further leverage genetic data, such as disease progression pathways, during model development.

As will be explained in greater detail below, the personalized progression module 150 may utilize the patient data in building a genetics-driven personalized disease progression model (GWA-PerDPM) (e.g., personalized disease progression model), the personalized disease progression model may be built separately for each of the patient groups associated with different inherent genetic markups. The inherent characteristics of patients that drive the personalized disease progression model differently may be defined by their inherent genetic compositions. In particular, the joint learning model where a patient's genetic grouping may be discovered from the GWAS along with developing the personalized disease progression models for each of those genetic groups. The personalized progression module 150 may first utilize a variational auto-encoder (VAE) which may find different groups of genetic clusters from the GWAS data. The personalized disease progression module 150 may then model the personalized disease progression models as a state-space model where the state representations are dependent upon the genetic markups, and clinical observation data, such as, prior treatment utilized and clinical history available thus far. The personalized disease progression model may be generic enough to discover the genetic groupings and their associated progression pathway from clinical observations, for example, by utilizing both static (e.g., genetic data) and longitudinal healthcare records, such as treatments, diagnostic variables measuring co-morbidities, and any clinical assessment of diseases such as laboratory measurements.

At 204, the personalized progression module 150 builds a personalized disease progression framework. The personalized disease progression framework may be utilized in learning different genetic markups corresponding to the plurality of patients for which data was received at step 202. As will be explained in more detail below and FIG. 5, the personalized progression module 150 may utilize a deep learning model based on a variational auto-encoder (VAE) in building the personalized disease progression framework.

The personalized disease progression framework may be a combined joint learning framework designed by the personalized progression module 150 to learn patient groupings and disease progression pathways. As will be explained in greater detail below, the personalized disease progression model may be trained using the personalized disease progression framework, wherein the one or more patient groupings are learned based on their respective genetic markers and corresponding personalized disease progression trajectory and/or pathway. In at least one embodiment, the personalized disease progression framework may be comprised of at least two parts. The first part may be the learning of patient groupings using the genetic data received at step 202 using the VAE and the second part may be the second part may be the utilization of the state-space technique for learning the personalized disease progression trajectory and/or pathway for each of the one or more patient groupings defined by the VAE. The personalized progression model may then leverage a joint learning framework to learn both the first and second part of the personalized disease progression framework.

The patient groupings may be defined by clusters of patients and the combined joint learning framework may learn the clusters and the disease progression pathways in each of these clusters (e.g., patient groupings). For example, let xᵢ∈ [symbol] ^{D} denote all the clinical features for a particular patient, i, where i∈{1, 2, . . . , N}, D and N represents a total number of clinical features and samples, respectively. Let X=[x₁; x₂; . . . , x_{N}]∈ [symbol] ^{N×D} denote the whole matrix of clinical observations received at step 202 from electronic health records (EHR). In this example, because EHR data may be observed at multiple irregular time points, we may utilize Xᵢ^{t} to denote the observed clinical features at some point t, where t∈{1, 2, . . . , Tᵢ} where, Tᵢ denotes a total number of visits made by the patient i. The clinical observations in this example may consist of temporal measurements, such as, but not limited to, lab assessments for the particular disease being modeled, image scans, etc. Here, let X∈ [symbol] ^{N×T×D} denote the final matrix of clinical observation containing all temporal data. Similarly, U∈^{R×X×T×B} may denote all the treatments collected temporarily where B may denote the dimensions of all possible treatments performed on the patients. Additionally, gᵢ∈ [symbol] ^{Q}, where M may be the total number of genetic features observed for the patient i. In this example, note that genetic features may be observed only during an initial visit of the patient, so G∈ [symbol] ^{N×Q}, may not have the temporal dimensions associated with them. Further, in this example, it may be assumed the whole population may be divided into K different groups, expressing different disease progression dynamics which may be implicitly encoded by genetic information, such that, the personalized disease progression module 150 may utilize a proxy variable, such as V∈ [symbol] ^{N×C}, to represent such groups. Accordingly, in this example, the task of the personalized progression module 150 may be to learn the latent representation V∈ [symbol] ^{N×h}, which may be utilized by the personalized progression module 150 in at least describing the hidden states of the disease progression.

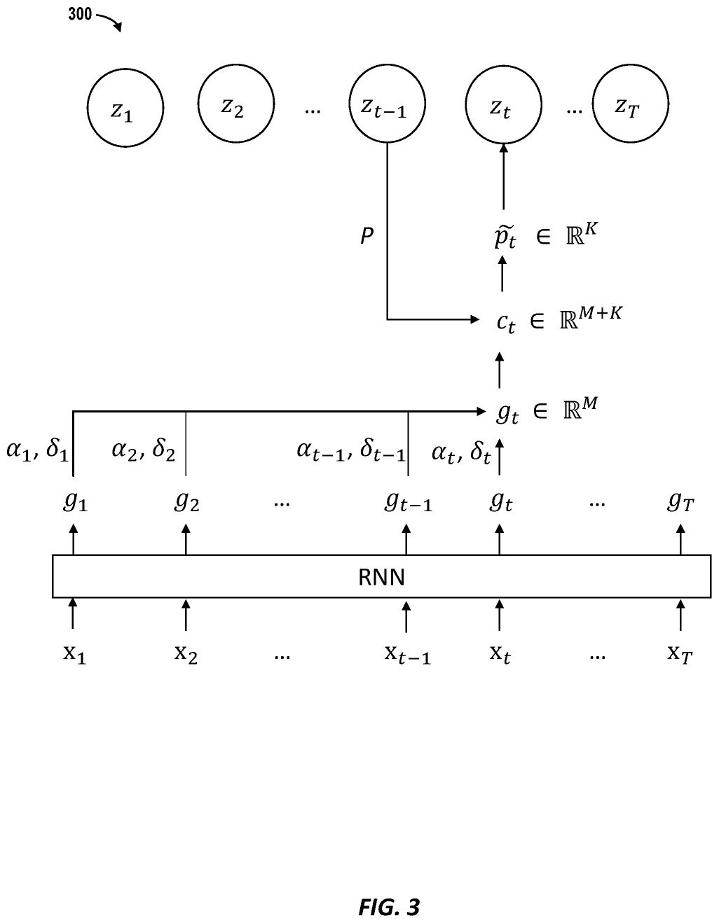

The personalized disease progression model may be a deep learning based generative Markov model, such as, but not limited to, a state-space model (SSM) for learning disease progression from large-scale data, such as the data received by the personalized progression module 150 at step 202. The neural network based SSM utilized by the personalized progression module 150 may best be described by continuing the example above in light of FIG. 3. Continuing with the above example, let xₜ=[x₁, x₂, . . . , xₜ] may represent the data for one subject and/or patient up to time t. The objective may be to model the disease progression and to assign a state to each time point. In this example, the personalized progression module 150 may start modeling the disease progression using a forward temporal model, which may be designed as a Recurrent Neural Network (RNN) to model the representation of the subject using the past information received at step 202 using the following equation:

[g₁, g₂, . . . , gₜ]=RNN (x₁, x₂, . . . , xₜ) ∀t {1, 2, . . . , T}

where gₜ∈ [symbol] ^{M} may be the forward representation of the subject and/or patient up to time t and M may be the dimension of the representation. However, one limitation of the model in the above equation may be that it assumes the data comes in regular times, which may not be a reality for the data received at step 202. Accordingly, the personalized progression module 150 may modify the model to include the time gap between two successive data points as:

$$= {\sum }_{i = 1}^{t} {α}_{i} {g}_{i} {δ}_{i}$$

the new representation [symbol] may be a linear combination of all representation prior to time t but it may put attention αᵢ to each time point i. These attentions may be learned during the model training so that the model may learn how important each time point may be to the current representation. δᵢ may be the time gap between the current and previous time points. In this example, the personalized progression module 150 may assume that there may be K states and P∈ [symbol] ^{K×K} may be the transition matrix. The state assignment at time t may be modeled by zₜ∈{1, 2, . . . , K}, which may be computed as a function using all the subject and/or patient information up to time t. In this example, under the Markovian assumption, pₜ^{˜k}=P(zₜ₋₁, k) may be the probability of transition from state zₜ₋₁ to state zₜ=k. The state assignment at time t may be modeled as:

$${c}_{t} = [ {\hat{g}}_{t} , {p}_{t}^{∼ 1} , {p}_{t}^{∼ 2} , … , {p}_{t}^{∼ K} ] ∈ {ℝ}^{M + K}$$

$$= softmax ( W {c}_{t} + b ) ∈ {ℝ}^{K}$$

where W∈ [symbol] ^{K×(M+K)} and Σₖ₌₁^{K} [symbol] =1. In which [symbol] may represent the probability of each state at time t. Finally, the state assignment may be modeled as a multimodal distribution over [symbol] , i.e., zₜ˜Multinominal ( [symbol] ). A graphical representation of a single disease progression model according to the example below may be described in more detail below with respect to at least FIG. 3. In this invention, the personalized progression module 150 extends the above described SSM based disease progression model which is also illustrated in FIG. 3 to using a genetics-driven personalized disease progression model (GWA-PerDPM) (e.g., personalized disease progression model).

For the personalized disease progression model, the personalized progression module 150 may utilize the transition between state Zs are conditioned on the genetic profile that may be learned from at least the GWA data received at step 202. In addition, the personalized progression module 150 may assume that the transition between states may be dependent on the treatment patterns such that the personalized disease progression model may be better characterized by the genetic markups of the plurality of patients for which data may be received at step 202.

The personalized disease progression framework models the genetic groupings from the available high-dimensional GWAS data using a variational auto-encoder, and then estimates the representations of the disease progression using the generative framework with the above-mentioned assumptions of progression being conditioned on these genetic markups and treatments. The generative model utilized for estimating genetic markups, the overall inference model for the disease progression model, the overall algorithm to learn both jointly, and how the proposed framework may be extended for supervised modeling where the state representations are known will all be described in greater detail below.

At 206, the personalized progression module 150 identifies one or more patient groupings. The personalized progression module 150 may identify the one or more patient groupings by filtering out one or more genes and the using static GWA data to define one or more clusters, wherein the one or more clusters correspond to the one or more patient groupings. The one or more clusters may be learned jointly in building the personalized disease progression framework through the VAE structure. Although, the identification of the one or more patient groupings and the determining of stages of the disease and the progression patterns are depicted and described as separate steps, the joint learning optimization framework for inference of multi-dimensional time-varying representation proposed may involve the clustering of genetic data and development of the corresponding disease progression model simultaneously.

At 208, the personalized progression module 150 determines stages of disease and progression patterns. The personalized progression module 150 may determine the stages of disease and the progression patterns for each of the one or more patient groupings identified at step 206. The one or more patient groupings may be heterogenous patient groups defined by genetics and the personalized disease progression model may define a progression pattern for a disease for each of the one or more patient groupings according to clinical observations as will be described in greater detail below.

The personalized progression module 150 may determine the stages of disease and the progression pattern using a genetics-driven personalized disease progression model (GWA-PerDPM) (e.g., personalized disease progression model) utilizing the personalized disease progression framework. While the personalized disease progression framework may be a generic framework which may be utilized for finding disease progression models for a wide range of chronic diseases the computational approaches of the personalized disease progression model based on whether the disease onset has occurred or not occurred. The personalized progression module 150 may define the personalized disease progression model based on clinical observations analyzed through the personalized disease progression framework. The stages of the of disease and progression patterns may be determined by the personalized disease progression model from clinical observations and GWA data. As will be explained in greater detail below, different algorithms may be utilized for determining disease progression through stages based on at least whether the disease onset is not given, unsupervised computational approaches, or the disease onset is given, supervised computational approaches. Utilizing evidence lower bound (ELBO) based algorithms and/or any other optimization framework, the personalized disease progression model may be flexible enough to be deployed in both unsupervised and supervised computational approaches for modeling disease progression. The joint learning framework may be utilized by the personalized progression module 150 in both finding inherent genetic proposition and/or disease progression patterns governed by genetic markups which may enable the personalized progression module 150 to identify different genetic groups that may drive diverse disease progression patterns.

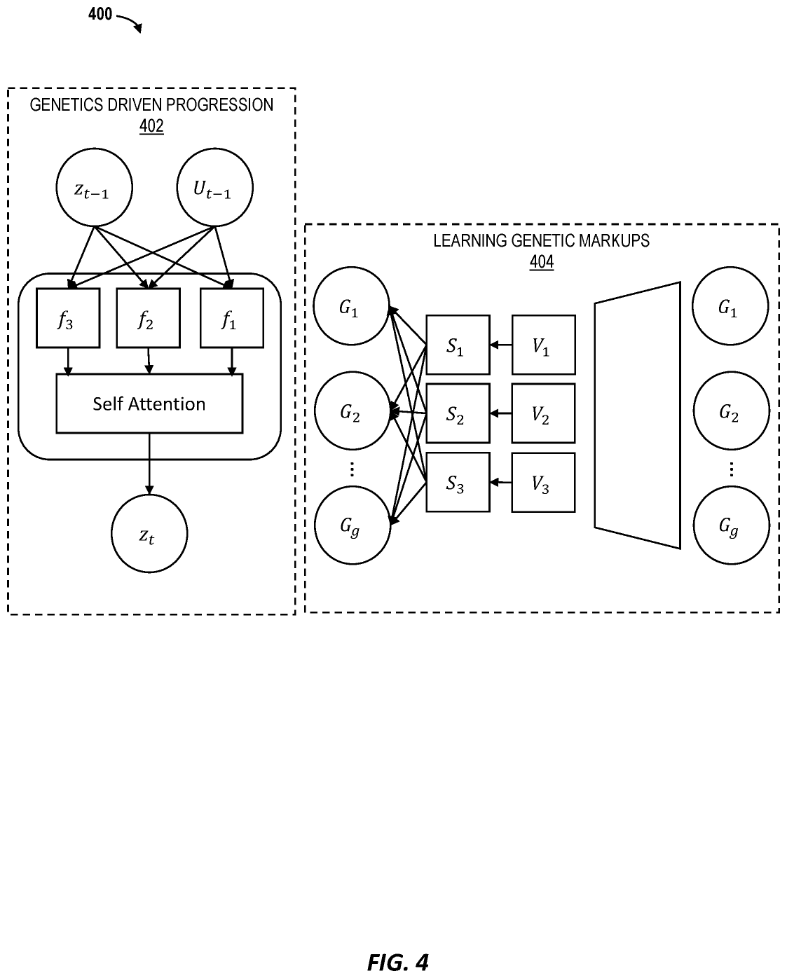

The personalized progression module 150 may learn the genetic markups using a deep-learning algorithm based on a variational auto-encoder (VAE) as will be described in greater detail below and with respect to FIG. 4. The deep learning model may be comprised of a Variational Auto-Encoder (VAE) and a SSM wherein genetic factors may be utilized as input. The genetic factors may be comprised of data from the GWA data received by the personalized progression module 150 at step 202, which may include genetic factors such as Single Nucleotide Polymorphisms (SNPs). The genetic data which may be collected in GWA studies may measure the genetic variation of an individual (e.g., patient) at a single base position of the individual's Deoxyribonucleic acid (DNA). These genetic variations, which may be referred to as Single Nucleotide Polymorphisms (SNPs), may impact how and/or to what degree a particular trait or disease phenotype may be manifested for an individual (e.g., patient) and to what degree. Additionally, the SNPs may have heterogeneous effects at different disease states or phases, some SNPs may potentially only have effects and some disease states or phases, but not an entire disease progression pathway, the SNPs may affect the transition intensity of the transition model, and the number of SNPs could be large for the GWAS dataset received at step 202 with only a few impacting the model. Accordingly, the VAE may be utilized in at least, finding genetic clusters, finding different genetic signatures by grouping the GWA data to lower dimensions using VAE based matrix factorization techniques, projecting the higher-dimensional SNPs to lower dimensions for better interpretability, and regularizing the input data to include existing domain knowledge, such as, but not limited to, pathways into account.

Accordingly, the deep learning model may be based on a VAE, in which the decoder of the VAE may be a one layer neural network, which may be parameterized by a weight matrix, for example, S∈ [symbol] ^{C×Q}, where C may represent the number of genetic groups, S decodes the distribution of genetic features for each cluster. The input of this module, the genetic factors, may be represented by G and the output of the module may be V, the clustering assignments of each sample to cluster c∈{1, 2, . . . , C}, and S as follows, wherein V=Encoder (G) and Ĝ=VS.

The genetic groupings and/or clusters learned using the VAE may be utilized by the state-space model, the genetic groupings discovered using the VAE may be heterogeneous patient groups defined by genetics. The disease progression pathways derived using the state-space model with attention framework may depend on the patient groupings, genetic groupings, and/or clusters learned from the VAE. As will be explained in more detail below, for each transition between two states, i and j, SNPs (Z) may have an impact. Given the assumptions of progression being conditioned on these genetic markups and treatments, the deep learning model may utilize a set of functions to model the genetics driven disease progressions. Each function may correspond to one genetic cluster and may take the corresponding genetic markups as input. The deep learning model may utilize attention mechanisms to learn the weight on different disease progression functions, and aggregate genetics driven disease progressions according to the learned weight. For example, the value and key may be the disease progression functions:

$$Value , Key = [ {f}_{1} ( {Z}_{t - 1} , {U}_{t - 1} , {S}_{1} ) , … , {f}_{p} ( {Z}_{t - 1} , {U}_{t - 1} , {S}_{p} ) ]$$

wherein f may represent the progression functions, Z may represent the latent variables (e.g., disease states), U may represent the interventional variables (e.g., medications), and S the inferred genetic statistic. In this example, the query may be the previous latent state, Query=Zₜ₋₁, wherein the transition process may be represented by the following equation:

$${Z}_{t} = W ( {\sum }_{i = 1}^{p} {softmax ( \frac{Query ⊙ Key}{\sqrt{h}} )}_{i} ⊙ {Value}_{i} ) + b$$

As will be explained in greater detail below with respect to cases in which the disease onset may not be unknown, not well measured, or staging may be disease onset agnostic which may utilize unsupervised computational approaches in the personalized disease progression model for modeling the disease progression using state-space model, i.e., at each time step t, patient is modeled to be in a state zₜ∈Z, which may be manifested in clinical observation Xₜ, we may assume the states are hidden and may be learned in an unsupervised fashion. The model proposed in the invention may be generic enough to be applicable for supervised modeling as well. As will be described in greater detail below in with respect to cases in which the disease onset is known and the invention may utilize supervised computational approaches in the personalized disease progression model the cross-entropy loss for predicted discrete states, [symbol] , and true states, Z, in addition to the original loss function.

In cases in which the disease onset may not be well measured or staging may be disease onset agnostic, the personalized progression module 150 may utilize unsupervised computational approaches in the personalized disease progression model. Examples of chronic diseases for which disease onset may not be well measured and/or staging may be disease onset agnostic may include, but is not limited to including, Parkinson's disease, Alzheimer Disease, Multiple Sclerosis, Schizophrenia, amongst many other diseases. In these examples, the personalized disease progression model may leverage unsupervised computational approaches, including, but not limited to including, Hidden Markov Model (HMM), Bayesian Networks, amongst other unsupervised computational approaches. As will be explained in greater detail below, the unsupervised model may utilize one or more maximum-likelihood detection algorithms, such as, but not limited to, the Viterbi algorithm, to learn the hidden disease stages and/or most likely sequence of hidden states of the HMM. In an embodiment for modeling the disease progression using state-space model, i.e., at each time step t, patient is modeled to be in a state zₜ∈Z, which may be manifested in clinical observation Xₜ, we may assume the states are hidden and may be learned in an unsupervised fashion.

In at least one embodiment, in which the states are hidden and may be learned in an unsupervised fashion, the personalized progression module 150 may model the joint distribution of states and observations using the Markovian assumption, amongst other approaches described above with may be utilized in SSM. Specifically, in this embodiment, the hidden variable Zₜ may depend only on the prior state Zₜ₋₁ and conditionally independent on any prior medical history since the prior history is captured by the previous states and treatment plan (Uₜ₋₁). In at least this embodiment, the generative process may be defined according to the below:

$$\begin{matrix}p ( {X}_{t} , G \vert {U}_{t} ) = {\int }_{z , v} p ( {X}_{t} , G \vert {Z}_{t} , {U}_{t} , V ) p ( {Z}_{t} , V \vert {Z}_{t - 1} , {U}_{t} ) dZdV \\ = {\int }_{Z , V} \{ p ( {X}_{t} \vert {Z}_{t} , {U}_{t} , V ) p ( G \vert {Z}_{t} , {U}_{t} , V ) p ( {Z}_{t} \vert {Z}_{t - 1} , \\ {U}_{t} , V ) p ( V \vert {U}_{t} ) dZdV \} \\ = {\int }_{Z , V} p ( {X}_{t} \vert {Z}_{t} ) p ( G \vert V ) p ( {Z}_{t} \vert {Z}_{t - 1} , {U}_{t} , V ) p ( V ) dZdV\end{matrix}$$

in the above embodiment, the personalized progression module 150 utilizes the joint distribution of Z and V to condition the previous latent state, which may be an assumption utilized in SSMs. Additionally, here, the personalized progression module 150 may assume the observations X may be conditionally independent of genetic information G given latent variables V, Z, and treatment U. Similarly, in this embodiment, the observations X may be conditionally independent of all other variables given latent state variables Z, the genetic information G may be conditionally independent of all other variables given the latent proxy variable V, and the latent proxy variable V may be independent of treatment U. According to the equation utilized above defining the generative process, the joint probability of all the observable variables may be:

$$p ( X , G , U ) = {π}_{t = 0}^{T} p ( {X}_{t} , G \vert {U}_{t} ) p ( {U}_{t} ) = {\int }_{Z , V} {π}_{t = 0}^{T} p ( {U}_{t} ) p ( {X}_{t} \vert {Z}_{t} ) p ( G \vert V ) p ( {Z}_{t} \vert {Z}_{t - 1} , {U}_{t} , V ) p ( V ) d Z d V$$

in the above equation, the personalized progression module 150 may utilize a special setting for the initial latent state, i.e., term p(Z₀|Uₜ, V), in at least assuring the edge case makes sense. Directly maximizing the likelihood shown in the above equation may be intractable, such that the personalized progression module 150 may learn utilizing maximizing a variational lower bound (ELBO). In this embodiment, the personalized progression module 150 may utilize Y=[X, G, U], and let D represent the dataset such that ELBO may be:

$$logp ( Y ) \geq {𝔼}_{Y ∼ D , q ϕ ( Z , V \vert Y )} [ logp ( Y \vert Z , V ) ] - {𝔼}_{Y ∼ D} [ K L ( {q}_{ϕ} ( Z , V \vert Y ) ‖ {p}_{θ} ( Z , V ) ) ]$$

where q₉₉ and p_{θ} may be learned posterior and prior distributions using the joint probability of the observable variables described above to expand yields of the above equation. Accordingly, the personalized progression module 150 may be:

$$ELBO = {𝔼}_{Y ∼ D , q ϕ ( Z , V \vert Y )} [ {\sum }_{t} ( log p ( {X}_{t} \vert {Z}_{t} ) + log p ( G \vert V ) ) ] - {𝔼}_{Y ∼ D} [ {\sum }_{t} K L ( {q}_{ϕ} ( {Z}_{t} , V \vert {Y}_{t} , {Z}_{t - 1} ) ‖ {p}_{θ} ( {Z}_{t} , V ) ) ]$$

accordingly, the personalized progression module 150 may further factorize ELBO into at least two components which may correspond to the VAE structure and the SSM according to the following:

$$log p ( Y ) \geq T {𝔼}_{Y ∼ D , q ϕ ( Z , V \vert Y )} [ log p ( G \vert V ) ] - T {𝔼}_{Y ∼ D} [ K L ( {q}_{ϕ} ( V \vert G ) ‖ {p}_{θ} ( V ) ) ] + {𝔼}_{Y ∼ D , q ϕ ( Z , V \vert Y )} [ {\sum }_{t} ( log p ( {X}_{t} \vert {Z}_{t} ) ] - {𝔼}_{Y ∼ D , q ϕ ( V \vert G )} [ K L ( {q}_{ϕ} ( {Z}_{t} \vert {Y}_{t} , {Z}_{t - 1} ) ‖ {p}_{θ} ( {Z}_{t} , V ) ) ]$$

wherein in the above equation, T [symbol] _{Y˜D,qϕ(Z,V|Y)}[logp(G|V)] and T [symbol] _{Y˜D}[KL(q_{ϕ}(V|G)∥p_{θ}(V))] may be modeled by the personalized progression module 150 using the VAE and [symbol] _{Y˜D,qϕ(Z,V|Y)}[Σₜ(logp (Xₜ|Zₜ)] and [symbol] _{Y˜D,qϕ(V|G)}[KL(q_{ϕ}(Zₜ|Yₜ, Zₜ₋₁)∥p_{θ}(Zₜ, V))] may be modeled by the personalized progression module 150 using the SSM.

In cases in which the disease onset may be measured and diseases progress through multiple stages, often from yielding from mild, to moderate, to severe stages, the personalized progression module 150 may utilize a supervised computational approach in the personalized disease progression model. Examples of diseases for which the disease onset is measured, and the disease progresses through multiple stages may include, but are not limited to including, cancer, diabetes, cardio-vascular diseases, amongst other diseases. In these examples, the personalized disease progression model may leverage supervised computational approaches, including, but not limited to including, Recurrent Neural Networks (RNNs), amongst other supervised computational approaches. In this embodiment, the personalized progression module 150 may incorporate the cross-entropy loss for predicted discrete states [symbol] and true states Z in addition to the original loss function described above based on the following equation:

$$log p ( Y ) \geq T {𝔼}_{Y ∼ D , q ϕ ( Z , V \vert Y )} [ log p ( G \vert V ) ] - T {𝔼}_{Y ∼ D} [ K L ( {q}_{ϕ} ( V \vert G ) ‖ {p}_{θ} ( V ) ) ] + {𝔼}_{Y ∼ D , q ϕ ( Z , V \vert Y )} [ {\sum }_{t} ( log p ( {X}_{t} \vert {Z}_{t} ) ] - {𝔼}_{Y ∼ D , q ϕ ( V \vert G )} [ K L ( {q}_{ϕ} ( {Z}_{t} \vert {Y}_{t} , {Z}_{t - 1} ) ‖ {p}_{θ} ( {Z}_{t} , V ) ) ]$$

The output of the personalized disease progression model may be similar regardless of whether an unsupervised computational approach or a supervised computational approach is utilized. As described in detail above, the primary difference between the two approaches utilized by the personalized progression module 150 may be the difference between the loss functions between the two types of models.

At 210, the personalized progression module 150 receives an output from the personalized disease progression model. The output received from the personalized disease progression model may include, but is not limited to including, personalized disease progression trajectories for each of the one or more patient groups, analysis of relationships between genetic data, such as, GWA data, and clinical observations, unique patient groupings and/or characteristics based on clustering.

The output for the personalized disease progression model may differ depending on whether an unsupervised computational approach or a supervised computational approach may be utilized. For example, for diseases in which the onset may be measured and the disease progresses through multiple stages the personalized disease progression model utilizing the supervised computational approach may include output data identifying a current stage of the disease and the personalized disease progression trajectories may include additional projections related to each of the multiple stages for each of the one or more corresponding patient groups. In this example, the personalized disease progression trajectory may be displayed to the user within the user interface for each of the one or more patient groupings and include at least a current stage and a progression pattern for each of the one or more patient groupings. Furthermore, as will be explained in greater detail below, the personalized progression module 150 may also display early intervention protocols and/or other recommendations to the user for each of the one or more patient groupings. For example, one of the outputs received from the personalized disease progression model may be the associations between the genetic markers and their corresponding disease progression patterns and/or disease trajectory patterns. This may enable the personalized progression module 150 to personalize the disease progression patterns and/or disease trajectory patterns in advance based on the genetic markup of the one or more patient groupings. Continuing with the above example, the presence of a particular genetic markup may accelerate the progression of the disease for a particular patient grouping. Accordingly, early interventions may be designed specific to the particular patient grouping to impede disease progress. These early interventions and/or other recommendations may be presented to the user in the user interface and the success of which may be monitored by the personalized progression module 150.

The personalized progression module 150 may also utilize the output described above in providing one or more recommendations to the user within the user interface. The one or more recommendations may include, but are not limited to including, early intervention protocols based on patient genetics and the corresponding disease progression pathway. The personalized progression module 150 may generate unique recommendations for each of the patient groupings based on the disease progression patterns corresponding to each patient grouping. Each of the recommendations may be presented to the user within the user interface and include additional information, such as, but not limited to, recommended treatment dosages, potential side effects, projected disease progression patterns associated with each of the one or more recommendations, amongst other information which may be displayed to the user within the user interface such that the user may make more informed decisions with respect to treatment.

The personalized progression module 150 may also monitor the accuracy of the output received from the personalized disease progression model as well as the effectiveness of the one or more recommendations derived from that output. For example, the personalized progression module 150 may continuously receive additional patient data, such as clinical observations recorded in additional practitioner visits. The additional patient data may be based on specific form documents generated by the personalized progression module 150 which include data entry options for specific continuous data which may further improve the personalized disease progression model. In this embodiment, the personalized progression module 150 may utilize the actual disease progression derived from the additional patient data in retraining and/or improving the personalized disease progression model to provide improved recommendations and more accurate output as additional patient data is received. The personalized progression module 150 may retrain and/or improve the personalized disease progression model by utilizing the actual disease progression derived in updating the personalized disease progression framework described at step 204. For example, the personalized disease progression module 150 may identify one or more sub groupings within the one or more patient groupings based on the additional patient data received, this may be utilized as feedback for the personalized disease progression framework such that patient groupings and their corresponding disease progression trajectories and/or pathways may be continuously learned and refined over time, enabling the personalized disease progression model to provide more detailed and accurate output according to more specific patient groupings which in turn may be utilized for more personalized early intervention protocols. The personalize progression module 150 may compare the actual disease progression derived from the additional patient data received to the personalized disease progression trajectory generated for the corresponding patient grouping. Furthermore, the personalized progression module 150 may revise the early intervention protocols for future recommendations.

The personalized progression module 150 may also utilize the output in discovering new and/or different disease progression pathways with potentially elucidating diverse biological mechanisms for a disease which may be present in one of the one or more patient groupings but not the other patient groupings.

Referring now to FIG. 3, a graphical representation 300 of a single disease progression model according to at least one embodiment is depicted.

The personalized disease progression model may be a deep learning based generative Markov model, such as, but not limited to, a state-space model (SSM) for learning disease progression from large-scale data, such as the data received by the personalized progression module 150 at step 202. The neural network based SSM utilized by the personalized progression module 150 may described by letting xₜ=[x₁, x₂, . . . , xₜ] represent the data for one subject and/or patient up to time t. The objective may be to model the disease progression and to assign a state to each time point. In this example, the personalized progression module 150 may start modeling the disease progression using a forward temporal model, which may be designed as a Recurrent Neural Network (RNN) to model the representation of the subject using the past information received at step 202 using the following equation:

[g₁, g₂, . . . , gₜ]=RNN (x₁, x₂, . . . , xₜ)∀t{1, 2, . . . , T}

where gₜ∈ [symbol] ^{M} may be the forward representation of the subject and/or patient up to time t and M may be the dimension of the representation. However, one limitation of the model in the above equation may be that it assumes the data comes in regular times, which may not be a reality for the data received at step 202. Accordingly, the personalized progression module 150 may modify the model to include the time gap between two successive data points as:

$$= {\sum }_{i = 1}^{t} {α}_{i} {g}_{i} {δ}_{i}$$

the new representation [symbol] may be a linear combination of all representation prior to time t but it may put attention αᵢ to each time point i. These attentions may be learned during the model training so that the model may learn how important each time point may be to the current representation. δᵢ may be the time gap between the current and previous time points. In this example, the personalized progression module 150 may assume that there may be K states and P∈ [symbol] ^{K×K} may be the transition matrix. The state assignment at time t may be modeled by zₜ∈{1, 2, . . . , K}, which may be computed as a function using all the subject and/or patient information up to time t. In this example, under the Markovian assumption, pₜ^{˜k}=P(zₜ₋₁, k) may be the probability of transition from state zₜ₋₁ to state zₜ=k. The state assignment at time t may be modeled as:

$${c}_{t} = [ {\hat{g}}_{t} , {p}_{t}^{∼ 1} , {p}_{t}^{∼ 2} , … , {p}_{t}^{∼ K} ] ∈ {ℝ}^{M + K}$$

$$= softmax ( W {c}_{t} + b ) ∈ {ℝ}^{K}$$

where W∈ [symbol] ^{K×(M+K)} and Σₖ₌₁^{K} [symbol] =1. In which [symbol] may represent the probability of each state at time t. Finally, the state assignment may be modeled as a multimodal distribution over [symbol] , i.e., zₜ˜Multinominal ( [symbol] ).

Referring now to FIG. 4, an architectural representation 400 of the personalized disease progression model according to at least one embodiment is depicted. In this embodiment, the personalized disease progression model may be comprised of at least Genetics Driven Progression 402 and Learning Genetic Markups 404.

In at least the architectural representation 400 depicted, f₁ may represent the progression functions, V₁ may represent inferred genetic clusters, S₁ may represent inferred genetic statistic, U may represent Interventional Variables, such as, but not limited to, medications, Z may represent Latent Variables, such as, but not limited to, disease states, X may represent Clinical Variables, such as, but not limited to Diagnosis Codes, and G may represent Genetic Markers, such as, but not limited to, SNPs. The architecture of the proposed model with respect to at least Genetics Driven Progression 402 and Learning Genetic Markups 404 will be described in greater detail below, additional details may be described above as part of the personalized disease progression modeling process 200.

With respect to Learning Genetic Markups 404, typically, the genetic data which may be collected in GWA studies may measure the genetic variation of an individual (e.g., patient) at a single base position of the individual's Deoxyribonucleic acid (DNA). These genetic variations, which may be referred to as Single Nucleotide Polymorphisms (SNPs), may impact how and/or to what degree a particular trait or disease phenotype may be manifested for an individual (e.g., patient) and to what degree. Additionally, the SNPs may have heterogeneous effects at different disease states or phases, some SNPs may potentially only have effects and some disease states or phases, but not an entire disease progression pathway, the SNPs may affect the transition intensity of the transition model, and the number of SNPs could be large for the GWAS dataset received at step 202 with only a few impacting the model. Accordingly, the VAE may be utilized in at least, finding genetic clusters, finding different genetic signatures by grouping the GWA data to lower dimensions using VAE based matrix factorization techniques, projecting the higher-dimensional SNPs to lower dimensions for better interpretability, and regularizing the input data to include existing domain knowledge, such as, but not limited to, pathways into account.

Accordingly, the deep learning model may be based on a VAE, in which the decoder of the VAE may be a one layer neural network, which may be parameterized by a weight matrix, for example, S∈ [symbol] ^{C×Q}, where C may represent the number of genetic groups, S decodes the distribution of genetic features for each cluster. The input of this module, the genetic factors, may be represented by G and the output of the module may be V, the clustering assignments of each sample to cluster c∈{1, 2, . . . , C}, and S as follows, wherein V=Encoder(G) and Ĝ=VS.

With respect to Genetics Driven Progression 402, given the assumptions of progression being conditioned on these genetic markups and treatments, the deep learning model may utilize a set of functions to model the genetics driven disease progressions. Each function may correspond to one genetic cluster and may take the corresponding genetic markups as input. The deep learning model may utilize attention mechanisms to learn the weight on different disease progression functions, and aggregate genetics driven disease progressions according to the learned weight. For example, the value and key may be the disease progression functions:

Value,Key=[f₁(Zₜ₋₁, Uₜ₋₁, S₁), . . . , fₚ(Zₜ₋₁, Uₜ₋₁, Sₚ)]

wherein f may represent the progression functions, Z may represent the latent variables (e.g., disease states), U may represent the interventional variables (e.g., medications), and S the inferred genetic statistic. In this example, the query may be the previous latent state, Query=Zₜ₋₁, wherein the transition process may be represented by the following equation:

$${Z}_{t} = W ( {\sum }_{i = 1}^{p} {softmax ( \frac{Query ⊙ Key}{\sqrt{h}} )}_{i} ⊙ {Value}_{i} ) + b$$

Referring now to FIG. 5, a deep learning model for genome-wide association data (GWA) utilized by the personalized progression module 150 within the personalized disease progression modeling process 200 is depicted.

The deep learning model may be comprised of a Variational Auto-Encoder (VAE) depicted within dashed box 504 and a State Space Model depicted within dashed box 506, wherein Genetic Factors 502 is utilized as input. The Genetic Factors 502 may be comprised of data from the GWA data received by the personalized progression module 150 at step 202, which may include genetic factors such as Single Nucleotide Polymorphisms (SNPs). The genetic data which may be collected in GWA studies may measure the genetic variation of an individual (e.g., patient) at a single base position of the individual's Deoxyribonucleic acid (DNA). These genetic variations, which may be referred to as Single Nucleotide Polymorphisms (SNPs), may impact how and/or to what degree a particular trait or disease phenotype may be manifested for an individual (e.g., patient) and to what degree. Additionally, the SNPs may have heterogeneous effects at different disease states or phases, some SNPs may potentially only have effects and some disease states or phases, but not an entire disease progression pathway, the SNPs may affect the transition intensity of the transition model, and the number of SNPs could be large for the GWAS dataset received at step 202 with only a few impacting the model. Accordingly, the VAE 504 may be utilized in at least, finding genetic clusters, finding different genetic signatures by grouping the GWA data to lower dimensions using VAE based matrix factorization techniques, projecting the higher-dimensional SNPs to lower dimensions for better interpretability, and regularizing the input data to include existing domain knowledge, such as, but not limited to, pathways into account.

Accordingly, the deep learning model 500 may be based on a VAE 504, in which the decoder of the VAE 504 may be a one layer neural network, which may be parameterized by a weight matrix, for example, S∈ [symbol] ^{C×Q}, where C may represent the number of genetic groups, S decodes the distribution of genetic features for each cluster. The input of this module, Genetic Factors 502, may be represented by G and the output of the module may be V, the clustering assignments of each sample to cluster c∈{1, 2, . . . , C}, and S as follows, wherein V=Encoder(G) and Ĝ=VS.

The genetic groupings and/or clusters learned using the VAE may be utilized by the State Space Model 506, the genetic groupings discovered using the VAE may be heterogeneous patient groups defined by genetics. The disease progression pathways derived using the State Space Model 506 with attention framework may depend on the patient groupings, genetic groupings, and/or clusters learned from the VAE. As will be explained in more detail below, for each transition between two states, i and j, SNPs (Z) may have an impact. Given the assumptions of progression being conditioned on these genetic markups and treatments, the deep learning model 500 may utilize a set of functions to model the genetics driven disease progressions. Each function may correspond to one genetic cluster and may take the corresponding genetic markups as input. The deep learning model 500 may utilize attention mechanisms to learn the weight on different disease progression functions, and aggregate genetics driven disease progressions according to the learned weight. For example, the value and key may be the disease progression functions:

Value,Key=[f₁(Zₜ₋₁, Uₜ₋₁, S₁), . . . , fₚ(Zₜ₋₁, Uₜ₋₁, Sₚ)]

wherein f may represent the progression functions, Z may represent the latent variables (e.g., disease states), U may represent the interventional variables (e.g., medications), and S the inferred genetic statistic. In this example, the query may be the previous latent state, Query=Zₜ₋₁, wherein the transition process may be represented by the following equation:

$${Z}_{t} = W ( {\sum }_{i = 1}^{p} {softmax ( \frac{Query ⊙ Key}{\sqrt{h}} )}_{i} ⊙ {Value}_{i} ) + b$$

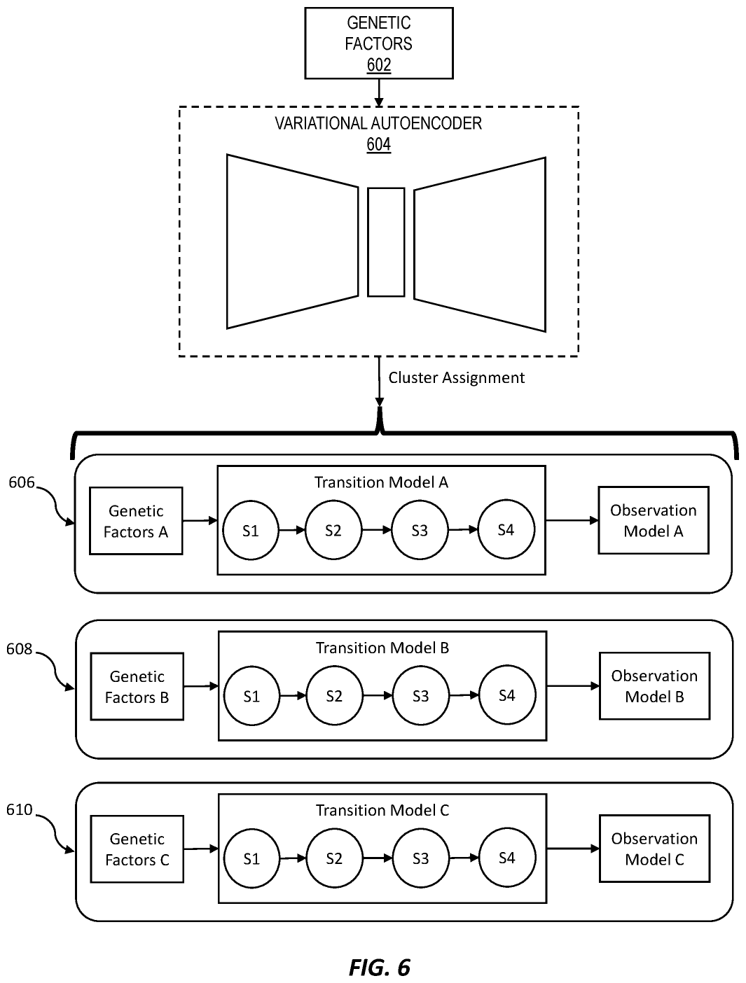

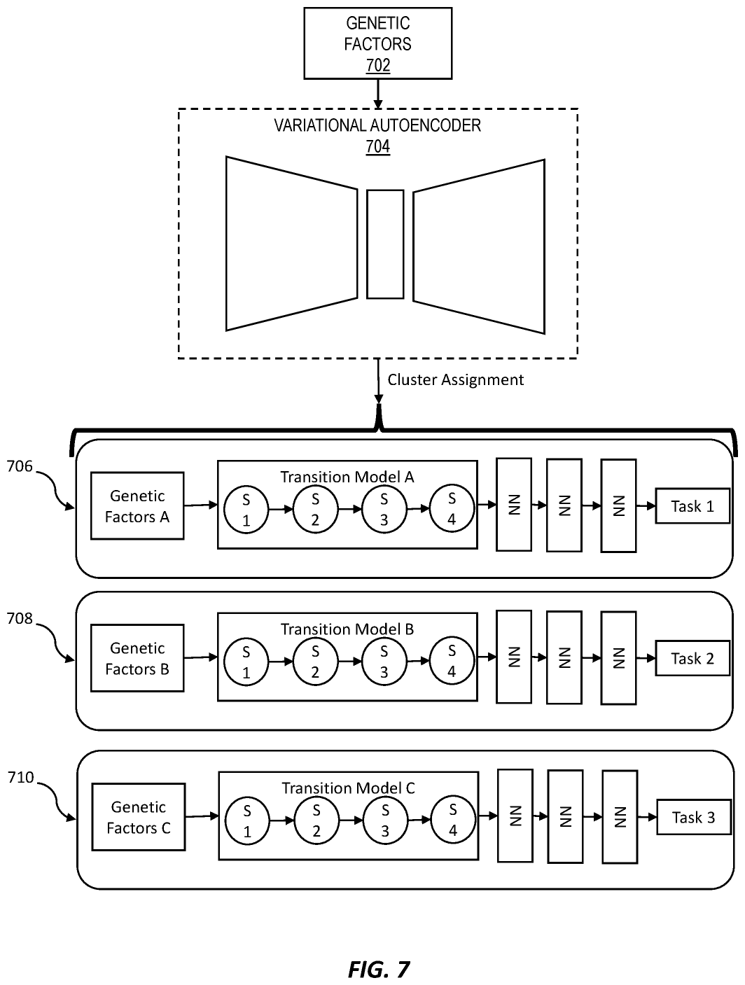

The state space model 506, illustrates two interventional variables, Medication 1 (e.g., Med 1) and Medication 2 (e.g., Med 2) which may be derived from the clinical observations described in greater detail at step 202. Additionally, the state space model 506 includes S1, S2, and S2, representing inferred genetic statistics and X1, X2, and X3, representing clinical variables (e.g., diagnosis code). As will be explained in greater detail below in FIG. 6 with respect to cases in which the disease onset may not be unknown, not well measured, or staging may be disease onset agnostic which may utilize unsupervised computational approaches in the personalized disease progression model for modeling the disease progression using state-space model, i.e., at each time step t, patient is modeled to be in a state zₜ∈Z, which may be manifested in clinical observation Xₜ, at FIG. 6 we may assume the states are hidden and may be learned in an unsupervised fashion. Additionally, as described throughout the personalized disease progression modeling process 200, the model proposed in the invention may be generic enough to be applicable for supervised modeling as well. As will be described in greater detail below in FIG. 7 with respect to cases in which the disease onset is known and the invention may utilize supervised computational approaches in the personalized disease progression model the cross-entropy loss for predicted discrete states, [symbol] , and true states, Z, in addition to the original loss function described at FIG. 6.

Referring now to FIG. 6, a personalized disease progression model which may be utilized by the personalized progression module 150 for embodiments in which a disease onset is unknown within the personalized disease progression modeling process 200 is depicted.

FIG. 6 depicts a personalized progression model which may utilize unsupervised computational approaches. Similar to FIG. 5, the personalized progression model depicted in this embodiment may utilize Genetic Factors 602 as input into a Variational Autoencoder 604 which may utilize a cluster assignment in producing the model output depicted in FIG. 6 using 606, 608, and 610. The personalized progression model using unsupervised computational approaches may be utilized in cases in which a disease onset may not be well measured and/or staging may be disease onset agnostic. Examples of chronic diseases for which disease onset may not be well measured and/or staging may be disease onset agnostic may include, but is not limited to including, Parkinson's disease, Alzheimer Disease, Multiple Sclerosis, Schizophrenia, amongst many other diseases. In these examples, the personalized disease progression model may leverage unsupervised computational approaches, including, but not limited to including, Hidden Markov Model, Bayesian Networks, amongst other unsupervised computational approaches.

In FIG. 6, 606 may correspond to Genetic Factors A, Transition Model A, and Observation Model A, while 608 may correspond to Genetic Factors B, Transition Model B, and Observation Model B, and 610 may correspond to Genetic Factors C, Transition Model C, and Observation Model C. The transition models is one which models the transitional probabilities between the states for the unsupervised model, and as described in FIG. 7 the supervised model, between the states. The unsupervised model, depicted in FIG. 6, has the additional task of learning the hidden disease stages, as opposed to the supervised model, depicted in FIG. 7. The unsupervised model may utilize one or more maximum-likelihood detection algorithms, such as, but not limited to, the Viterbi algorithm, to learn the hidden disease stages and/or most likely sequence of hidden states in a Hidden Markov Model (HMM), as described in greater detail above with respect to the disease progression modeling process 200. The output of the personalized disease progression model may be similar regardless of whether an unsupervised computational approach or a supervised computational approach is utilized. As described in detail above with respect to the disease progression modeling process 200, the primary difference between the two approaches utilized by the personalized progression module 150 may be the difference between the loss functions between the two types of models.

Referring now to FIG. 7, a personalized disease progression model which may be utilized by the personalized progression module 150 for embodiments in which the disease onset is known within the personalized disease progression modeling process 200 is depicted.

FIG. 7 depicts a personalized progression model which may utilize supervised computational approaches. Similar to FIG. 5, the personalized progression model depicted in this embodiment may utilize Genetic Factors 702 as input into a Variational Autoencoder 704 which may utilize a cluster assignment in producing the model output depicted in FIG. 7 using 706, 708, and 710. The personalized progression model using the supervised computational approaches may be utilized in cases in which the disease onset may be measured, and the disease progresses through multiple stages, often from yielding from mid, to moderate, to severe stages. Examples of diseases for which the disease onset is measurable, and the disease progresses through multiple stages may include, but are not limited to including, cancer, diabetes, cardiovascular diseases, amongst others. In these examples, the personalized disease progression model may leverage supervised computational approaches, including, but not limited to including, Recurrent Neural Networks (RNNs), amongst other supervised computational approaches.

In FIG. 7, 706 may correspond to Genetic Factors A, Transition Model A, and Task A, while 708 may correspond to Genetic Factors B, Transition Model B, and Task B, and 710 may correspond to Genetic Factors C, Transition Model C, and Task C. The “NN” utilized in 706, 708, and 710, represent the Neural Network described above. Task 1, Task 2, and Task 3 may represent the known stages of the disease, which may be utilizes as a loss function as described in greater detail above which respect the personalizes disease progression modeling process 200.

It may be appreciated that FIG. 2-7 provide only an illustration of one embodiment and do not imply any limitations with regard to how different embodiments may be implemented. Many modifications to the depicted embodiment(s) may be made based on design and implementation requirements.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of one or more transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

The present disclosure shall not be construed as to violate or encourage the violation of any local, state, federal, or international law with respect to privacy protection.

What is claimed is:

1. A method for unsupervised disease progression modeling, the method comprising:
receiving patient data, wherein the patient data is comprised of at least genetic data and clinical observations;
building a personalized disease progression framework using one or more patient groupings and one or more disease progression pathways derived from the patient data; and
determining a disease progression pattern for each of the one or more patient groupings using a personalized disease progression model, wherein the personalized disease progression model utilizes unsupervised computational approaches in leveraging the personalized disease progression framework.

2. The method of claim 1, wherein the genetic data includes genome-wide association study (GWAS) data.

3. The method of claim 2, wherein the one or more patient groupings are determined by clustering the GWAS data of a plurality of patients.

4. The method of claim 1, further comprising:
displaying, in a user interface, the disease stage progression pattern for each of the one or more patient groupings using a personalized disease progression trajectory for each of the one or more patient groupings.

5. The method of claim 4, further comprising:
monitoring an actual disease progression pattern based on additional patient data received; and
comparing the actual disease progression pattern to the personalized disease progression trajectory for a corresponding patient grouping.

6. The method of claim 5, further comprising:
retraining the personalized disease progression model to improve an accuracy of future personalized disease progression trajectories for new patient data.

7. The method of claim 4, further comprising:
updating the personalized disease progression trajectory based on the additional patient data received.

8. The method of claim 1, further comprising:
generating one or more recommendations for each of the one or more patient groupings based on the disease progression pattern corresponding to each patient grouping, wherein the one or more recommendations includes at least a set of early intervention protocols.

9. The method of claim 8, further comprising:
monitoring an effectiveness of the one or more recommendations including the set of early intervention protocols; and
revising the early intervention protocols for future recommendations.

10. A computer system for unsupervised disease progression modeling, comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
program instructions, stored on at least one of the one or more computer-readable storage media for execution by at least one of the one or more processors via at least one of the one or more memories, to receive patient data, wherein the patient data is comprised of at least genetic data and clinical observations;
program instructions, stored on at least one of the one or more computer-readable storage media for execution by at least one of the one or more processors via at least one of the one or more memories, to build a personalized disease progression framework using one or more patient groupings and one or more disease progression pathways derived from the patient data; and
program instructions, stored on at least one of the one or more computer-readable storage media for execution by at least one of the one or more processors via at least one of the one or more memories, to determine a disease progression pattern for each of the one or more patient groupings using a personalized disease progression model, wherein the personalized disease progression model utilizes unsupervised computational approaches in leveraging the personalized disease progression framework.

11. The computer system of claim 10, wherein the genetic data includes genome-wide association study (GWAS) data.

12. The computer system of claim 10, further comprising:
program instructions, stored on at least one of the one or more computer-readable storage media for execution by at least one of the one or more processors via at least one of the one or more memories, to display, in a user interface, the disease stage progression pattern for each of the one or more patient groupings using a personalized disease progression trajectory for each of the one or more patient groupings.

13. The computer system of claim 12, further comprising:
program instructions, stored on at least one of the one or more computer-readable storage media for execution by at least one of the one or more processors via at least one of the one or more memories, to monitor monitoring an actual disease progression pattern based on additional patient data received; and
program instructions, stored on at least one of the one or more computer-readable storage media for execution by at least one of the one or more processors via at least one of the one or more memories, to compare the actual disease progression pattern to the personalized disease progression trajectory for a corresponding patient grouping.

14. The computer system of claim 13, further comprising:
program instructions, stored on at least one of the one or more computer-readable storage media for execution by at least one of the one or more processors via at least one of the one or more memories, to retrain the personalized disease progression model to improve an accuracy of future personalized disease progression trajectories for new patient data.

15. The computer system of claim 10, further comprising:
program instructions, stored on at least one of the one or more computer-readable storage media for execution by at least one of the one or more processors via at least one of the one or more memories, to generate one or more recommendations for each of the one or more patient groupings based on the disease progression pattern corresponding to each patient grouping, wherein the one or more recommendations includes at least a set of early intervention protocols.

16. A computer program product for unsupervised disease progression modeling, comprising:
one or more computer readable storage media, and program instructions collectively stored on the one or more computer readable storage media, the program instructions comprising:
program instructions, stored on at least one of the one or more computer-readable storage media, to receive patient data, wherein the patient data is comprised of at least genetic data and clinical observations;
program instructions, stored on at least one of the one or more computer-readable storage media, to build a personalized disease progression framework using one or more patient groupings and one or more disease progression pathways derived from the patient data; and
program instructions, stored on at least one of the one or more computer-readable storage media, to determine a disease progression pattern for each of the one or more patient groupings using a personalized disease progression model, wherein the personalized disease progression model utilizes unsupervised computational approaches in leveraging the personalized disease progression framework.

17. The computer program product of claim 16, wherein the genetic data includes genome-wide association study (GWAS) data.

18. The computer program product of claim 16, further comprising:
program instructions, stored on at least one of the one or more computer-readable storage media, to display, in a user interface, the disease stage progression pattern for each of the one or more patient groupings using a personalized disease progression trajectory for each of the one or more patient groupings.

19. The computer program product of claim 18, further comprising:
program instructions, stored on at least one of the one or more computer-readable storage media, to monitor an actual disease progression pattern based on additional patient data received; and
program instructions, stored on at least one of the one or more computer-readable storage media, to compare the actual disease progression pattern to the personalized disease progression trajectory for a corresponding patient grouping.

20. The computer program product of claim 19, further comprising:
program instructions, stored on at least one of the one or more computer-readable storage media, to retrain the personalized disease progression model to improve an accuracy of future personalized disease progression trajectories for new patient data.

21. A method for supervised disease progression modeling, the method comprising:
receiving patient data, wherein the patient data is comprised of at least genetic data and clinical observations;
building a personalized disease progression framework using one or more patient groupings and one or more disease progression pathways derived from the patient data; and
determining a personalized disease progression trajectory for each of the one or more patient groupings using a personalized disease progression model.

22. The method of claim 21, wherein the personalized disease progression trajectory includes at least identifying a current stage from a plurality of disease stages and a progression pattern for each of the one or more patient groups.

23. The method of claim 21, wherein the personalized disease progression model utilizes supervised computational approaches in leveraging the personalized disease progression framework.

24. A computer system for supervise disease progression modeling, comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
program instructions, stored on at least one of the one or more computer-readable storage media for execution by at least one of the one or more processors via at least one of the one or more memories, to receive patient data, wherein the patient data is comprised of at least genetic data and clinical observations;
program instructions, stored on at least one of the one or more computer-readable storage media for execution by at least one of the one or more processors via at least one of the one or more memories, to build a personalized disease progression framework using one or more patient groupings and one or more disease progression pathways derived from the patient data; and
program instructions, stored on at least one of the one or more computer-readable storage media for execution by at least one of the one or more processors via at least one of the one or more memories, to determine a personalized disease progression trajectory for each of the one or more patient groupings using a personalized disease progression model.

25. The computer system of claim 24, wherein the personalized disease progression trajectory includes at least identifying a current stage from a plurality of disease stages and a progression pattern for each of the one or more patient groups.
